(19) United States
(12) Patent Application Publication
(10) Pub. No.: US 2026/0191640 A1
(43) Pub. Date: Jul. 9, 2026

(54) COMMISSURE ATTACHMENT FOR A PROSTHETIC HEART VALVE

(71) Applicant: Edwards Lifesciences Corporation, Irvine, CA (US)

(72) Inventors: Yara Khader, Haifa (IL); Anatoly Dvorsky, Haifa (IL)

(21) Appl. No.: 19/559,354

(22) Filed: Mar. 6, 2026

Related U.S. Application Data
(63) Continuation of application No. PCT/US2024/046146, filed on Sep. 11, 2024.
(60) Provisional application No. 63/582,375, filed on Sep. 13, 2023.

(51) Int. Cl. A61F 2/24

(52) U.S. Cl. CPC A61F 2/2418; A61F 2220/0075; A61F 2230/0006

(57) ABSTRACT

Commissure features and attachments for prosthetic heart valves are disclosed. As one example, a prosthetic heart valve can include an annular frame comprising a plurality of commissure features, each commissure feature comprising a first sidewall strut, a second sidewall strut, and a central strut situated between the first and second sidewall struts. A plurality of leaflets is arranged within an interior of the frame and secured to one another at adjacent ends to form commissures, each commissure being secured to a respective commissure feature and comprising a first commissure tab of a first leaflet and a second commissure tab of a second leaflet adjacent the first leaflet. The first commissure tab and the second commissure tab each extend inside of the central strut, wrap around a respective sidewall strut, and extend into the interior of the frame.

### CROSS-REFERENCE TO RELATED APPLICATIONS

This application is a continuation of PCT Patent Application No. PCT/US2024/046146 filed on September 11, 2024, which application claims priority to and the benefit of U.S. Provisional Patent Application No. 63/582,375, filed on September 13, 2023, each of these applications being incorporated by reference herein in its entirety.

### FIELD

The present disclosure relates to expandable prosthetic heart valves, including commissure attachments for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Most expandable, prosthetic heart valves comprise a radially expandable and compressible cylindrical metal frame or stent and prosthetic leaflets mounted inside the frame. Leaflet commissures can be formed by connecting pairs of commissure tabs of adjacent leaflets to each other, which are then connected to commissure windows or commissure supports of the frame. Current techniques for properly securing a leaflet commissure to a frame, which involve the formation of multiple stitches that pass through the commissure tabs and portions of the frame, are extremely time consuming and require a great deal of skill and precision. Accordingly, improving and simplifying the attachment between commissures and the frame of a prosthetic heart valves is desirable.

### SUMMARY

Described herein are prosthetic heart valves, delivery apparatuses, and methods for implanting prosthetic heart valves. In particular, described herein are examples of commissure features and attachments for prosthetic heart valves. As such, the devices and methods disclosed herein can, among other things, overcome one or more of the deficiencies of typical prosthetic heart valves.

A prosthetic heart valve can comprise a frame and a valve structure coupled to the frame. In addition to these components, a prosthetic heart valve can further comprise one or more components disclosed herein.

In some examples, a prosthetic heart valve can comprise a sealing member configured to reduce paravalvular leakage.

In some examples, a prosthetic heart valve frame can comprise a commissure feature comprising a first sidewall strut, a second sidewall strut, and a central strut situated between the first and second sidewall struts.

In some examples, a prosthetic heart valve can comprise a leaflet commissure comprising first and second commissure tabs, each commissure tab extending inside of the central strut of the commissure feature, wrapping around a respective sidewall strut of the commissure feature, and extending into the interior of the frame.

In some examples, a prosthetic heart valve can comprise first and second commissure tabs connected to each other outside of the frame.

In some examples, a prosthetic heart valve can comprise first and second commissure tabs connected to each other within the interior of the frame.

In some examples, a prosthetic heart valve comprises an annular frame comprising a plurality of commissure features, each commissure feature comprising a first sidewall strut, a second sidewall strut, and a central strut situated between the first and second sidewall struts; and a plurality of leaflets arranged within an interior of the frame and secured to one another at adjacent ends to form commissures, each commissure being secured to a respective commissure feature and comprising a first commissure tab of a first leaflet and a second commissure tab of a second leaflet adjacent the first leaflet, wherein the first commissure tab and the second commissure tab each extend inside of the central strut, wrap around a respective sidewall strut, and extend into the interior of the frame.

In some examples, a prosthetic heart valve comprises an annular frame comprising a plurality of interconnected struts, the plurality of interconnected struts defining a plurality of axially extending commissure features, each commissure feature comprising a first axial strut, a second axial strut, and a central axial strut situated between the first and second axial struts, wherein the first and central axial struts define a first commissure window and the second and central axial struts define a second commissure window; and a plurality of leaflets arranged within an interior of the frame and secured to one another at adjacent ends to form commissures, wherein each commissure is secured to a respective commissure feature by extending laterally across an interior surface of the central axial strut, through the first and second commissure windows, along an exterior surface of the first and second axial struts, and into the interior of the frame.

In some examples, a prosthetic heart valve comprises an annular frame comprising a plurality of interconnected struts, the plurality of interconnected struts defining a plurality of adjacent pairs of first and second commissure windows; and a plurality of leaflets arranged within an interior of the frame and secured to one another at adjacent ends to form commissures, each commissure secured to a respective pair of first and second commissure windows by extending outwardly through the first and second commissure windows, around a pair of outer edges of the first and second commissure windows from an exterior of the frame, and inwardly into an interior of the frame from the outer edges of the first and second commissure windows, wherein each commissure comprises a first leaflet tab of one leaflet and a second leaflet tab of an adjacent leaflet secured to one another along first and second stitch lines outside of the frame and a third stitch line inside of the frame.

In some examples, a prosthetic heart valve comprises one or more of the features recited in Examples 1-54 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of a frame of the prosthetic heart valve of FIG. 1.

FIG. 3 is a side view of an exemplary delivery apparatus configured to deliver and implant a radially expandable prosthetic heart valve at an implantation site.

FIG. 4 is a perspective view of one of the leaflets of the prosthetic heart valve of FIG. 1 shown in a flattened configuration.

FIG. 5 is an enlarged side view of a pair of commissure windows, according to one example, that can be included in an alternative example of the frame and prosthetic heart valve of FIGS. 1-2.

FIG. 6 is a top-down cross-sectional view of the commissure windows of FIG. 5 and a top plan view of a commissure formed by two adjacent leaflets secured to the commissure windows.

FIG. 7 is an enlarged side view of the commissure and commissure windows of FIG. 6 viewed from outside the frame.

FIG. 8 is an enlarged side view of the commissure and commissure windows of FIGS. 5-6, taken along a radius of the frame.

FIG. 9 is a top-down plan view of a commissure, according to another example.

FIG. 10 is an enlarged side view of a commissure and commissure windows, according to another example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient’s body), while distal motion of the device is motion of the device away from the user and toward the implantation site (e.g., into the patient’s body). The terms “longitudinal” and “axial” refer to an axis extending in the proximal and distal directions, unless otherwise expressly defined. As used herein, “e.g.” means “for example,” and “i.e.” means “that is.”

### Overview of the Disclosed Technology

As introduced above, prosthetic heart valves can include leaflets that are secured to one another at their adjacent sides (e.g., commissure tabs) to form commissures which are secured to a frame of the prosthetic heart valve. In some instances, each commissure can extend through a commissure window in the frame of the prosthetic heart valve and be secured to the struts of the frame. Typically, securing the commissures to the frame can be arduous and time consuming, often taking several sutures and stitches passing through and around the struts of the frame defining the commissure window and/or other portions of the frame.

Described herein are examples of prosthetic heart valves that can include a frame having one or more pairs of adjacent commissure windows in which commissures of adjacently paired leaflets of the prosthetic heart valve can be secured in a simplified manner. Each pair of adjacent commissure windows can be defined by a pair of outer sidewall struts and a central inner strut situated between the sidewall struts. The commissure tabs of a single commissure, in some examples, can overlap each other and extend inside of the central strut, wrap around a respective sidewall strut, and extend into the interior of the frame. In-and-out stitches can be used to secure the commissure tabs to each other outside of the frame and inside of the frame.

In some examples, a pair of commissure tabs can be sutured to each other along a pair of stitch lines outside of the frame and along another stitch line within the interior of the frame. The commissures, in such examples, are secured to a respective pair of commissure windows by extending laterally across an interior surface of the central strut, through both commissure windows, along an exterior surface of the sidewall struts, and into the interior of the frame. Attachment between the commissures and commissure windows in this manner can, in some examples, allow the leaflets to be mounted to the frame without, or in a limited fashion, stitching the commissures to the struts of the frame directly.

### Examples of the Disclosed Technology

The prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

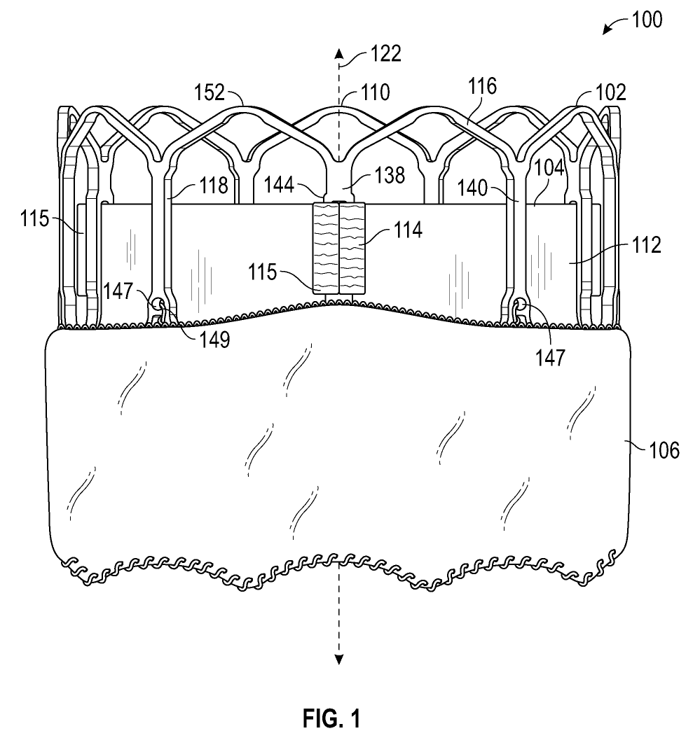

FIG. 1 shows a prosthetic heart valve 100 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

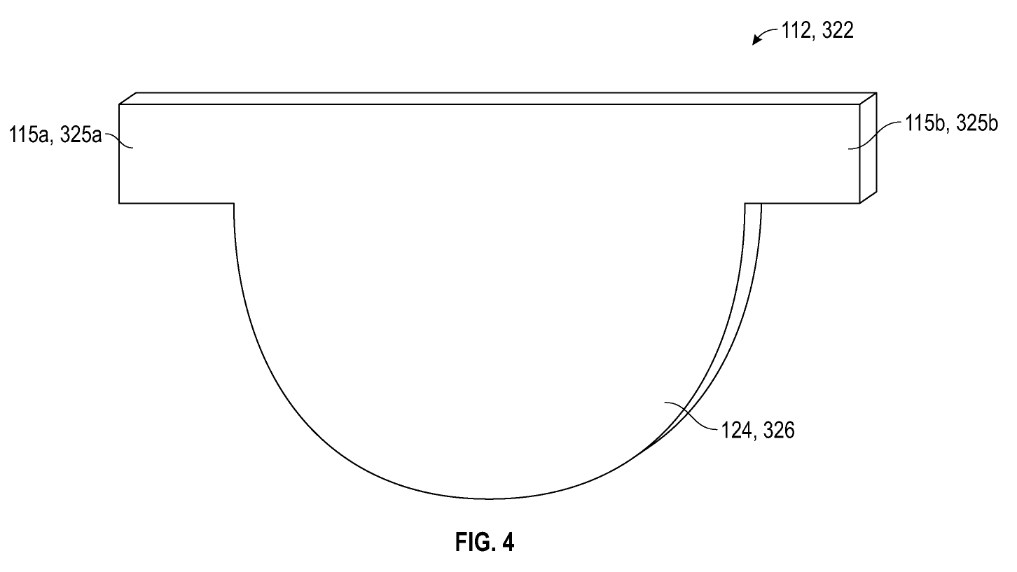

The valvular structure 104 can comprise a plurality of leaflets 112 (e.g., three leaflets, as shown in FIG. 1), collectively forming a leaflet structure, which can be arranged to collapse in a tricuspid arrangement. The valvular structure 104 can, for example, be configured to regulate the flow of blood through the prosthetic valve 100 from the inflow end 108 to the outflow end 110. The leaflets 112 can be secured to one another at their adjacent sides (e.g., commissure tabs) to form commissures 114 of the valvular structure 104. For example, FIG. 4 shows that each leaflet 112 can comprise opposing commissure tabs 115a, 115b disposed on opposite sides of the leaflet 112 and a cusp edge portion 124 extending between the opposing commissure tabs 115. The cusp edge portion 124 of the leaflets 112 can have an undulating, curved scalloped shape, and can be secured directly to the frame 102 (e.g., by sutures). However, in alternate examples, the cusp edge portion 124 of the leaflets 112 can be secured to an inner skirt which is then secured to the frame 102. In some examples, the leaflets 112 can be formed of pericardial tissue (e.g., bovine pericardial tissue), biocompatible synthetic materials, or various other suitable natural or synthetic materials as known in the art and described in U.S. Patent No. 6,730,118, which is incorporated by reference herein.

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to the angled struts 134. Examples of such outer skirts, as well as various other outer skirts, that can be used with the frame 102 can be found in the provisional patent application under Edwards attorney docket No. 12131US01, which is incorporated by reference herein.

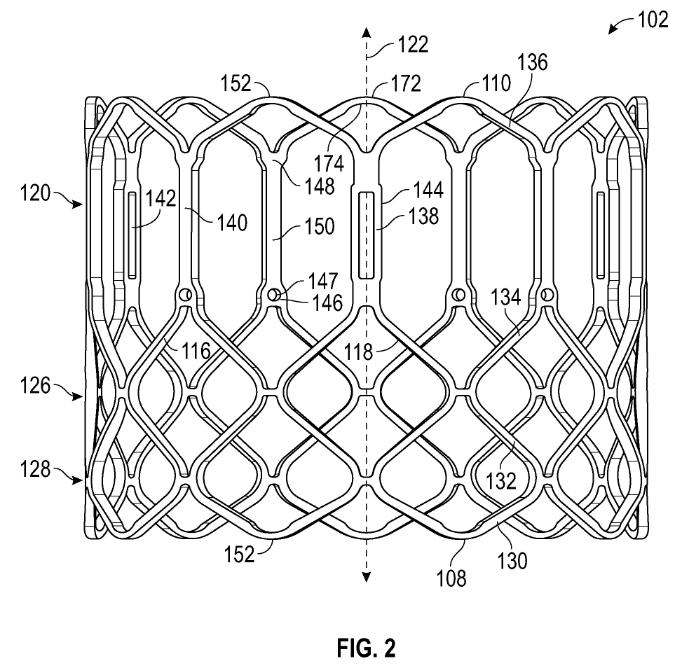

The frame 102 can be radially compressible and expandable between a radially compressed configuration and a radially expanded configuration (the expanded configuration is shown in FIG. 1). The frame 102 is shown alone in FIG. 2.

The frame 102 can be made of any of various suitable plastically expandable materials (e.g., stainless steel, etc.) or self-expanding materials (e.g., nickel titanium alloy (NiTi), such as nitinol). When constructed of a plastically expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically expandable materials that can be used to form the frame 102 include, without limitation, stainless steel, metal alloys, polymers, or combinations thereof. Example metal alloys can comprise one or more of the following: nickel, cobalt, chromium, molybdenum, titanium, or other biocompatible metal. As an example, a nickel-based alloy, such as a cobalt-chromium or a nickel-cobalt-chromium alloy can be used. In particular examples, frame 102 can be made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIG. 2, the frame 102 can comprise a plurality of interconnected struts 116 which form multiple rows of open cells 118 between the outflow end 110 and the inflow end 108 of the frame 102. In some examples, the frame 102 can comprise three rows of cells 118 with a first row of cells 120 disposed at the outflow end 110 (the upper row in the orientation shown in FIG. 2). The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length than cells 118 in the remaining rows of cells, which can include a second row of cells 126 and a third row of cells 128, the third row of cells 128 disposed at the inflow end 108 and the second row of cells 126 disposed between the first row of cells 120 and the third row of cells 128.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (e.g., four or five) and/or more or less than nine cells per row. In some examples, the cells 118 in the first row of cells 120 may not be elongated compared to cells 118 in the remaining rows of cells of the frame 102 (that is, the second row of cells 126 and the third row of cells 128).

The interconnected struts 116 can include a plurality of angled struts 130, 132, 134, and 136 arranged in a plurality of rows of circumferentially or laterally extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102.

The fourth row of angled struts 136 can be connected to the third row of angled struts 134 by a plurality of axially extending commissure features 144 and a plurality of axial struts 140 (e.g., axially extending). The commissure features 144 can comprise axially extending window struts 138 (or window strut portions) that define commissure windows 142 (e.g., open windows). The plurality of commissure features 144 can be spaced apart from one another around the frame 102, in a circumferential direction, and used to receive a pair of commissure tabs 115 of a pair of adjacent leaflets 112 (FIG. 4) arranged into a commissure 114 (FIG. 1). Each commissure feature can be said to be configured to receive and/or be secured to a pair of commissure tabs of a pair of adjacent leaflets.

One or more axial struts 140 (e.g., two, as shown in FIG. 2) can be positioned between, in the circumferential direction, two commissure windows 142 formed by the window struts 138. Since the frame 102 can include fewer cells per row (e.g., nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 extends from a location defined by the convergence of the lower ends (e.g., ends arranged inward of and farthest away from the outflow end 110) of two angled struts 136 (which can also be referred to as an upper strut junction or upper elongated strut junction) to another location defined by the convergence of the upper ends (e.g., ends arranged closer to the outflow end 110) of two angled struts 134 (which can also be referred to as a lower strut junction or lower elongate strut junction). Each axial strut 140 and each window strut 138 forms an axial side of two adjacent cells of the first row of cells 120.

Commissure tabs 115 (FIG. 4) of adjacent leaflets 112 can be secured together to form commissures 114 (FIG. 1). Each commissure 114 of the prosthetic heart valve 100 comprises two commissure tabs 115a, 115b paired together, one from each of two adjacent leaflets 112, and extending through a commissure window 142 of the frame 102. Each commissure 114 can be secured to the window struts 138 forming the commissure window 142.

The cusp edge portion 124 (e.g., scallop edge) of each leaflet 112 can be secured to the frame 102 via one or more fasteners (e.g., sutures). In some examples, the cusp edge portion 124 of each leaflet 112 can be secured directly to the struts of the frame 102 (e.g., angled struts 130, 132, and 134). For example, the cusp edge portions 124 of the leaflets 112 can be sutured to the angled struts 130, 132, and 134 that generally follow the contour of the cusp edge portions 124 of the leaflets 112.

In some examples, the cusp edge portion 124 of the leaflets 112 can be secured to an inner skirt and the inner skirt can then be secured directly to the frame 102.

As shown in FIG. 2, in some examples, one or more of or each of the axial struts 140 can comprise an inflow end portion 146 (e.g., an end portion that is closest to the inflow end 108) and an outflow end portion 148 that are widened relative to a middle portion 150 of the axial strut 140. In some instances, the inflow end portion 146 of the axial strut 140 can comprise an aperture 147. The apertures 147 can be configured to receive fasteners (e.g., sutures) for attaching soft components of the prosthetic heart valve 100 to the frame 102. For example, in some instances, the outer skirt 106 can be positioned around the outer surface of the frame 102 and an upper or outflow edge portion of the outer skirt 106 can be secured to the apertures 147 by fasteners 149 (e.g., sutures), as shown in FIG. 1.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

Additional details and examples of frames for prosthetic heart valves that include apex regions can be found in U.S. Provisional Patent Application Nos. 63/178,416, filed April 22, 2021, 63/194,830, filed May 28, 2021, 63/279,096, filed November 13, 2021, and 63/342,269, filed May 16, 2022, all of which are incorporated by reference herein.

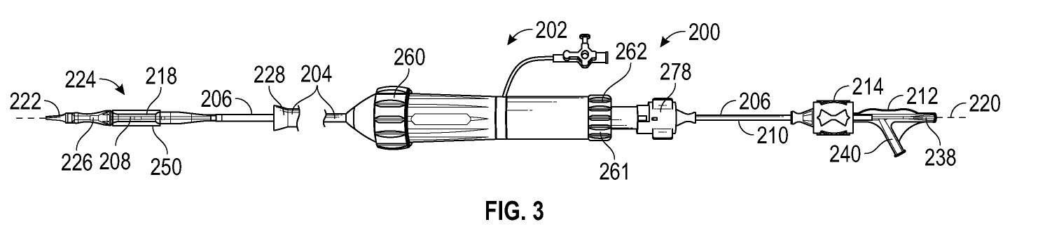

FIG. 3 shows a delivery apparatus 200, according to an example, that can be used to implant an expandable prosthetic heart valve (e.g., the prosthetic heart valve 100 of FIG. 1 and/or any of the other prosthetic heart valves described herein). In some examples, the delivery apparatus 200 is specifically adapted for use in introducing a prosthetic valve into a heart.

The delivery apparatus 200 in the illustrated example of FIG. 3 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (e.g., move) longitudinally, along a central longitudinal axis 220 of the delivery apparatus 200, relative to one another to facilitate delivery and positioning of a prosthetic valve at an implantation site in a patient’s body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (e.g., inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 3), or to an alternate component at the distal end of the delivery apparatus 200 (e.g., a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 3, a prosthetic heart valve 250 (which can be one of the prosthetic valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient’s vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 3) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Patent No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (e.g., for fine positioning at the implantation site). Further details on the delivery apparatus 200 can be found in PCT Application No. PCT/US2021/047056, which is incorporated by reference herein.

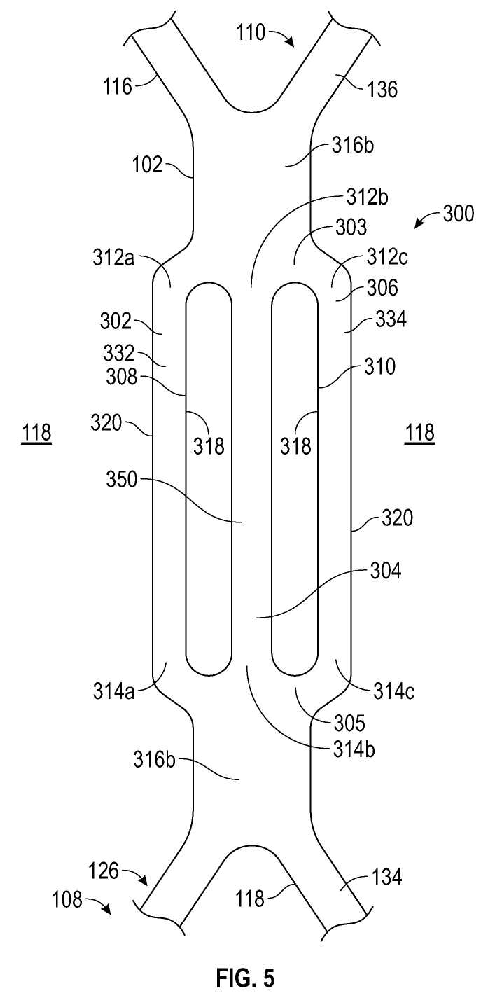

FIG. 5 shows one alternate example of a commissure feature 300 that can be a portion of the frame 102 of FIGS. 1 and 2. The commissure feature 300 shown in FIG. 5, for example, can be substituted for any one or all of the commissure features 144 (for example, window struts 138 and commissure windows 142) described above. As such, the frame 102 can include one or more commissure features 300 (for example, three commissure features 300 and three corresponding commissures) in addition to any combination of the components and features already described in connection with FIGS. 1 and 2, a description of which is not repeated here for brevity.

As shown in FIG. 5, each commissure feature 300 can comprise three axially extending window struts 302, 304, 306. Specifically, each commissure feature 300 can include a pair of outer sidewall struts 302, 306 and an inner central strut 304 situated between the sidewall struts 302, 306. The outer sidewall struts 302, 306 and central strut 304 can be circumferentially aligned and spaced from one another along a circumference of the frame 102, such that the struts 302, 304, 306 lie along the same circumferential plane.

The sidewall struts 302, 306 and central strut 304 can be connected at respective upper ends 312a, 312b,312c and respective lower ends 314a, 314b, 314c. As shown in FIG. 5, the ends of the struts 302, 304, 306 nearest the outflow end 110 of the frame 102 can be connected by a first lateral strut 303 (or lateral strut segment) and define the upper ends 312a-312c. The ends of the struts 302, 304, 306 nearest the inflow end 108 of the frame 102 can be connected by a second lateral strut 305 (or lateral strut segment) and define the lower ends 314a-314c.

The commissure features 300 can connect adjacent rows of circumferentially extending interconnected struts 116. As one example, such as that shown in FIG. 5, the third and fourth rows of circumferentially and laterally extending angled struts 134, 136 can be connected by one or more commissure features 300. In particular, the upper ends 312a-312c of the struts 302, 304, 306 can be connected to the angled struts 136 at the outflow end 110 of the frame 102 and the lower ends 314a-314c of the struts 302, 304, 306 can be connected to the angled struts 134 defining the outflow end of a respective cell 118 situated along the second row of cells 126 (FIG. 2). In some examples, the commissure features 300 can positioned at other locations along the height of the frame and can connect adjacent first and second rows of angled struts, adjacent second and third rows of angled struts, adjacent fourth and fifth rows of angled struts, etc.

In some examples, a first vertical or axial strut segment 316a can connect and extend between the upper ends 312a-312c and the angled struts 136. A second vertical or axial strut segment 316b can also connect and extend between the lower ends 314a-314c and the angled struts 134. For instance, the first axial strut segment 316a can connect the upper ends 312a-312c to an upper strut junction defined at the ends of the angled struts 136 arranged inward of and furthest away from the outflow end 110 of the frame 102. Similarly, the second axial strut segment 316b can connect the lower ends 314a-314c to a lower strut junction defined at the ends of the angled struts 134 arranged closer to the outflow end 110 of the frame 102. In some examples, outermost edges 320 of the sidewall struts 302, 306 define an axial side of two adjacent cells 118 of the first row of cells 120.

As shown in FIG. 5, the sidewall struts 302, 306 and central strut 304 can define a pair of adjacent commissure windows 308, 310. For example, each commissure window 308, 310 can be defined by a central strut 304, a respective sidewall strut 302, 306, and the closed curved surfaces defined at the upper and lower ends 312, 314 by the lateral struts 303, 305. In this configuration, the sidewall struts 302, 306 and central strut 304 can be said to define a pair of fully enclosed commissure windows 308, 310. That is, an inner edge 318 of each commissure window 308, 310 can be continuous along a lateral and longitudinally extending edge of a corresponding sidewall strut 302, 306, along the curved surfaces at the upper and lower ends 312, 314, and along a lateral and longitudinally extending edge of the central strut 302. While described as fully enclosed, it should be appreciated that one or more openings can be defined along the inner edge 318 of one or both commissure windows 308, 310. As one example, an opening can extend through the upper end 312 or the lower end 314 of one or both of the outer sidewall struts 302, 306, to an outermost edge 320 and into an adjacent cell 118.

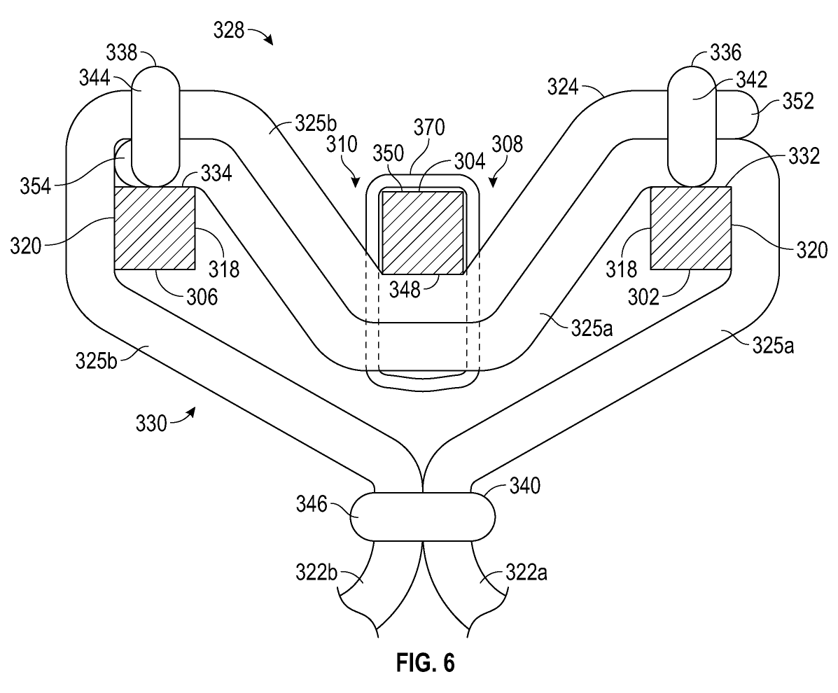

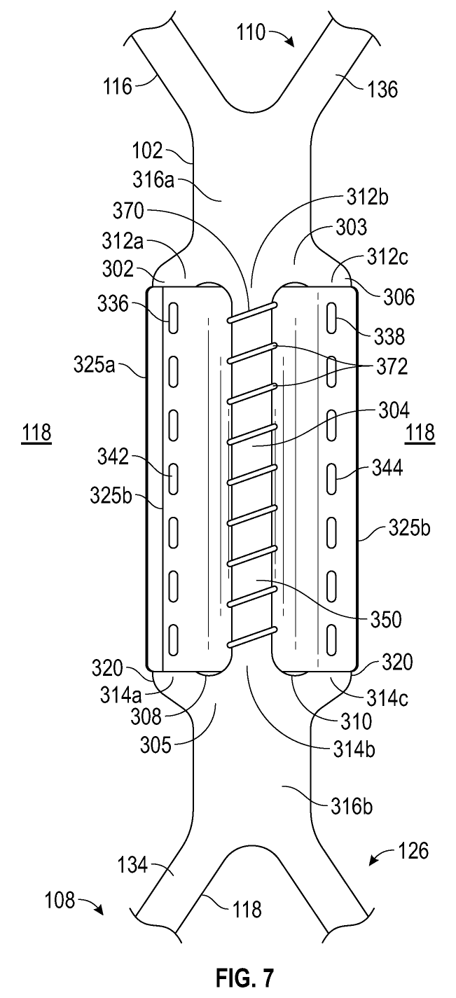

As shown in FIGS. 6-7, the sidewall struts 302, 306 and central strut 304 can be equal in length and be circumferentially spaced from each other along a circumference of the frame 102 (in the left and right directions in FIGS. 5-7) such that each commissure window 308, 310 is sized and shaped to receive commissure tabs of adjacent leaflets. Specifically, FIG. 6 shows a top-down cross-sectional view of a single commissure feature 300 and a top plan view of a respective leaflet commissure 324 secured to the commissure feature 300. The leaflet commissure 324 can be formed from a pair of commissure tabs 325a, 325b of adjacent leaflets 322a, 322b (FIG. 4) forming a valvular structure (e.g., a valvular structure similar to valvular structure 104). FIG. 7 shows a side view of the commissure 324 and commissure feature 300 viewed from outside of the frame 102.

FIGS. 6-7 show that the leaflets 322a, 322b can be mounted within the frame 102 by way of attachment between the commissure feature 300 and commissure 324. The leaflets 322a, 322b can, for example, be used to construct the prosthetic heart valve 100. That is to say, when the frame 102 comprises the commissure feature 300, the leaflets 322 and commissure 324 can be substituted for the leaflets 112 and commissures 114 to construct the prosthetic heart valve 100 described above. As such, when comprising the commissure feature 300, leaflets 322, and commissure 324, the prosthetic heart valve 100 can have the same functionality and include any or all other features and components described above in connection to FIGS. 1 and 2.

While not expressly shown, it should be appreciated that the leaflets 322, while mounted within the frame 102, can form a valvular structure and be arranged to collapse in a tricuspid arrangement (e.g., similarly to valvular structure 104). As shown in FIG. 4, the leaflets 322 can also include each of the features described in connection with leaflets 112, including opposing commissure tabs 325a, 325b disposed on opposite sides of the leaflet 322 and a cusp edge portion 326 extending between the opposing commissure tabs 325. The cusp edge portion 326 can have an undulating, curved scalloped shape, and can be secured directly to the frame 102 (e.g., by sutures) and/or secured to an inner skirt which is then secured to the frame 102. The leaflets 322 can be formed of pericardial tissue (e.g., bovine pericardial tissue), biocompatible synthetic materials, or various other suitable natural or synthetic materials as described herein.

As shown in FIGS. 6-7, each commissure 324 can be formed of a pair of commissure tabs 325a, 325b of adjacent leaflets 322a, 322b. To form a commissure 324, for instance, a first commissure tab 325a of the first leaflet 322a can be wrapped around an outermost edge 320 of a first sidewall strut 302, wrapped around an exterior surface 332 of the first sidewall strut 302 on the outside 328 of the frame 102, and advanced through a first commissure window 308 defined by the central strut 304 and first sidewall strut 302. The first commissure tab 325a can be advanced laterally in a circumferential direction relative to and on the inside 330 of the frame 102 such that the commissure tab 325a extends across the central strut 304 on the inside 330 of the frame 102 and can be advanced and extend through a second commissure window 310. The second commissure window 310 can be adjacent the first window 308 and defined by the central strut 304 and second sidewall strut 306. A portion of the commissure tab 325a extending through the second window 310 and to the outside 328 of the frame 102 can be positioned along at least a portion of an exterior surface 334 of the second sidewall strut 306. In this configuration, at least a portion of the commissure tab 325a can be said to be in contact, directly or indirectly, with the exterior surface 334 of the second sidewall strut 306 and the outermost edge 320 and exterior surface 332 of the first sidewall strut 302.

In a similar manner, the second commissure tab 325b of the second leaflet 322b can be wrapped around an outermost edge 320 and the exterior surface 334 of the second sidewall strut 306. The second commissure tab 325b can also be wrapped around the portion of the first commissure tab 325a along the exterior surface 334 of the second sidewall strut 306. The commissure tab 325b can be advanced through the second commissure window 310 between the first commissure tab 325a and the central strut 304 such that the commissure tab 325b extends laterally in a circumferential direction across the central strut 304 on the inside 330 of the frame 102. The commissure tab 325b can be advanced and extend through the adjacent first commissure window 308 to the outside 328 of the frame 102. The portion of the commissure tab 325b extending along the outside 328 of the frame 102 can be positioned along the first commissure tab 325a and extend circumferentially across (e.g., parallel to) at least a portion of the exterior surface 332 of the first sidewall strut 302. Positioned in this way, at least a portion of the second commissure tab 325b can be said to be in contact, directly or indirectly, with the outermost edge 320 of the second sidewall strut 306, an interior surface 348 of the central strut 304, and the first commissure tab 325a along the first sidewall strut 302.

FIG. 6 shows that the portions of the commissure tabs 325a, 325b wrapped around and outside of the outermost edges 320 of the first and second sidewall struts 302, 306 can be oriented and positioned to extend inwardly into the frame 102. The first and second commissure tabs 325a, 325b, for instance, can be oriented such that the commissure tabs 325a, 325b can be secured to each other on the inside 330 of the frame 102. For example, the first commissure tab 325a of the first leaflet 322a that extends along the outermost edge 320 of the first sidewall strut 302 can be oriented to extend inwardly into the frame 102 to position the first commissure tab 325a proximate and/or in contact with the second commissure tab 325b of the second leaflet 322b. In the same fashion, the second commissure tab 325b of the second leaflet 322b that extends along the outer most edge 320 of the second sidewall strut 306 can be oriented to extend inwardly into the frame 102 to position the second commissure tab 325b proximate and/or in contact with the first commissure tab 325a.

As shown in FIGS. 6-8, to fully form and secure the commissure 324 to the commissure feature 300, the first and second commissure tabs 325a, 325b of the leaflets 322a, 322b can be secured to each other along advantageously placed stitch lines (or suture lines) at locations inside and outside of the frame 102. For example, the commissure tabs 325a, 325b can be connected to each other along first and second stitch lines 336, 338 on the outside 328 of the frame 102 and a third stitch line 340 on the inside 330 the frame 102. First and second sets of fasteners 342, 344 (for example, sutures) can form in-and-out stitches extending through the first and second commissure tabs 325a, 325b along the first and second stitch lines 336, 338 to secure the first and second commissure tabs 325a, 325b to each other on the outside 328 of the frame 102. The suture lines 336, 338, as shown in FIGS. 6-7, can be proximate and extend parallel to the first and second sidewall struts 302, 306, respectively. As such, the first and second stitch lines 336, 338 can be said to extend in a vertical or axial direction relative to the frame, that is, extend in the direction of the inflow and outflow ends 108, 110 of the frame 102. The first and second stitch lines 336, 338 can also be parallel to the longitudinal axis 122 of the frame 102.

As shown in FIG. 7, the first and second stitch lines 336, 338 can, in some instances, extend the entire axial length or a substantial portion of the axial length of the commissure 324. In some examples, such as illustrated in FIG. 6, the first and second stitch lines 336, 338 can be radially aligned with the first and second sidewall struts 302, 306, respectively (for example, relative to the longitudinal axis 122 of the frame 102). In some examples, one or both of the stitch lines 336, 338 need not be radially aligned with a respective sidewall strut 302, 306.

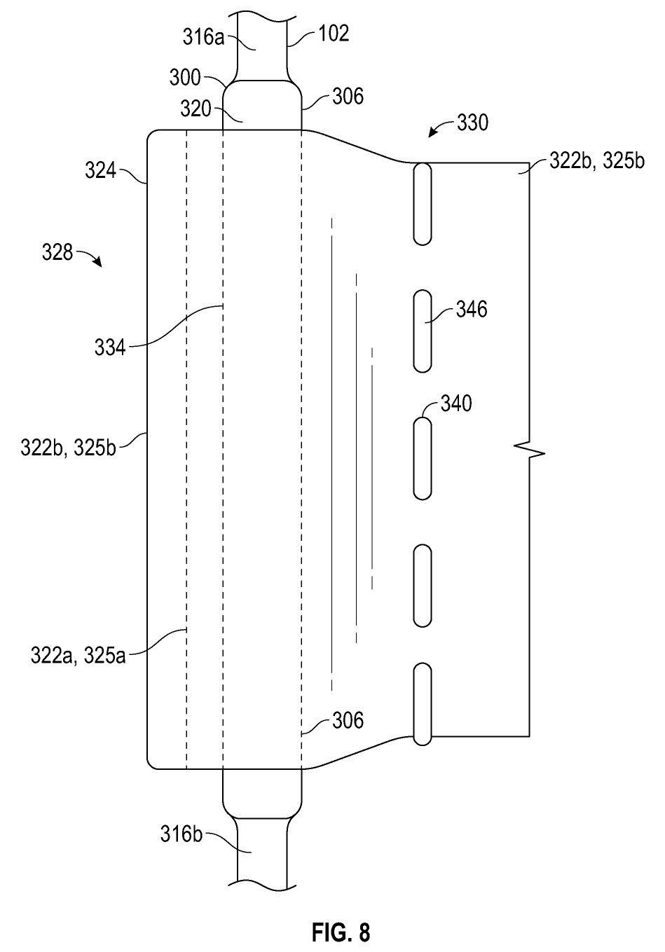

FIG. 8 shows a side view of the commissure 324 and commissure feature 300 taken along a radius of the frame 102 (for example, from within an adjacent cell 118 looking along the circumference of the frame 102). As shown in FIGS. 6 and 8, a third set of fasteners 346 (e.g., sutures) can form in-and-out stitches extending through the first and second commissure tabs 325a, 325b along the third stitch line 340 to secure the first and second commissure tabs 325a, 325b to each other on the inside 330 of the frame 102. FIG. 8 shows that the third stitch line 340 can extend in a vertical or axial direction parallel to the commissure feature 300 (e.g., parallel to the central strut 304) and the longitudinal axis 122 of the frame 102. The third stitch line 340 can also extend the entire or a substantial portion of the axial length of the commissure 324. FIG. 6 shows that, in some examples, the third stitch line 340 can be radially aligned with the central strut 304 relative to a longitudinal axis 122 of the frame 102. The main bodies of the leaflets 322a, 322b can articulate (bend) at the stitch line 340 as they move between open and closed positions to regulate the flow of the blood through the prosthetic valve. In some examples, the third stitch line 340 need not be radially aligned with the central strut 304.

Consequently, the first and second stitch lines 336, 338 can be said to be circumferentially spaced apart and aligned with each other due to their positioning relative to the first and second sidewall struts 302, 306, while the third stitch line 340 can be said to be in neither circumferential alignment nor in radial alignment with the first and second stitch lines 336, 338. The third stitch line 340 can also be positioned more inwardly relative to the longitudinal axis 122 of the frame 102 (for example, inside 330 the frame 102) than the first and second stitch lines 336, 338 positioned on the outside 328 of the frame 102.

The first and second commissure tabs 325a, 325b of the commissure 324 can radially overlap each other laterally across the commissure feature 300 on both the inside and the outside of the frame 102. For instance, FIGS. 6 and 8 show the first and second commissure tabs 325a, 325b can overlap each other between the first and second stitch lines 336, 338. Specifically, the first and second commissure tabs 325a, 325b can overlap each other from the first stitch line 336 to the second stitch line 338, through the first and second commissure windows 308, 310, and circumferentially or laterally across the central strut 304. As shown in FIGS. 6-8, those portions of the first and second commissure tabs 325a, 325b at or nearest the first and second stitch lines 336, 338 and extending across the exterior surfaces 332, 334 of the outer sidewall struts 302, 306 radially overlap each other on the outside 328 of the frame 102. The first commissure tab 325a, in this way, can be situated between the second commissure tab 325b and the exterior surfaces 332, 334 of the outer sidewall struts 302, 306.

At or approximately midway between the first stitch line 336 and the second stitch line 338, the first and second commissure tabs 325a, 325b can radially overlap each other on the inside 330 of the frame 102. For instance, the first and second commissure tabs 325a, 325b can radially overlap each other where the commissure tabs 325a, 325b extend circumferentially or laterally across the interior surface 348 of the central strut 304. The second commissure tab 325b, in this way, can be situated between the first commissure tab 325a and the interior surface 348 of the central strut 304. Because the commissure 324 extends circumferentially across the commissure feature 300, the first and second commissure tabs 325a, 325b can also be said to radially overlap over the length of the commissure 324 extending between the first stitch line 336 and the second stitch line 338.

As shown in FIG. 6, the first and second commissure tabs 325a, 325b can also circumferentially overlap each other on the inside 330 of the frame 102. In particular, the portions of the first and second commissure tabs 325a, 325b secured to each other at the third stitch line 340 on the inside 330 of the frame 102 can overlap each other in a lateral or circumferential direction (e.g., in the left and right directions in FIG. 6). As such, the first and second commissure tabs 325a, 325b of the commissure 324 can be said to radially overlap each other outside 328 the frame 102 and radially and circumferentially overlap each other inside 330 the frame 102.

The overlapping portions of the first and second commissure tabs 325a, 325b extending through first and second commissure windows 308, 310 can extend through the windows at a non-zero angle relative to the circumferential plane of the commissure feature 300. In particular, the overlapping portions of the first and second commissure tabs 325a, 325b extending between the interior surface 348 of the central strut 304 and the exterior surfaces 332, 334 of the first and second sidewall struts 302, 306, do so at an angle. That is, the overlapping portions of the first and second commissure tabs 325a, 325b extending through the commissure windows 308, 310 can be neither in circumferential alignment nor perpendicular to the circumferential plane of the struts 302, 304, 306.

In some examples, the first and second commissure tabs 325a, 325b can partially overlap each other between the first stitch line 336 and the third stitch line 340 and/or between the second stitch line 338 and the third stich line 338. For example, a portion 352 of the second commissure tab 325b can extend beyond the first stitch line 336 in the direction of the outermost edge 320 of the first sidewall strut 302 to partially overlap the first commissure tab 325b. In a like manner, a portion 354 of the first commissure tab 325a can extend beyond the second stitch line 338 in the direction of the outermost edge 320 of the second sidewall strut 306 and partially overlap with the second commissure tab 325b along the exterior surface 334 and/or outermost edge 320 of the second sidewall strut 306. In some examples, the first and second commissure tabs 325a, 325b only overlap between the first stitch line 336 and second stitch line 338, and not between the third stitch 340 and either of the first and second stitch lines 336, 338.

As shown in FIG. 6, certain aspects of the commissure feature 300 can either be situated on the outside of the commissure 324 or fully enclosed within the commissure 324 once the commissure 324 is fully formed. For example, since the first and second commissure tabs 325a, 325b extend across the central strut 304 on the inside 330 the frame 102, through the first and second commissure windows 308, 310, and around the first and second sidewall struts 302, 306, the central strut 304 is situated on the outside of the commissure 324 (FIG. 7). In this instance, an exterior surface 350 of the central strut 304 is exposed on the outside 328 of the frame 102 (FIGS. 6-7). In contrast, the outer sidewall struts 302, 306 are fully enclosed within the commissure 324 due to the first and second commissure tabs 325a, 325b extending laterally across the commissure feature 300 and extending inwardly and being secured on the inside 330 of the frame 102.

As shown in FIGS. 6 and 7, the commissure 324 can include an optional stitch line 370 that secures the commissure tabs 325a, 325b directly to the central strut 304. The stitch line 370 can be formed by one or more fasteners (for example, sutures) forming one or more loop or whip stitches 372, each of which extends around the strut 304 and through the commissure tabs 325a, 325b. The stitches 372 can be continuous stitches or discrete stitches formed by separate pieces of suture.

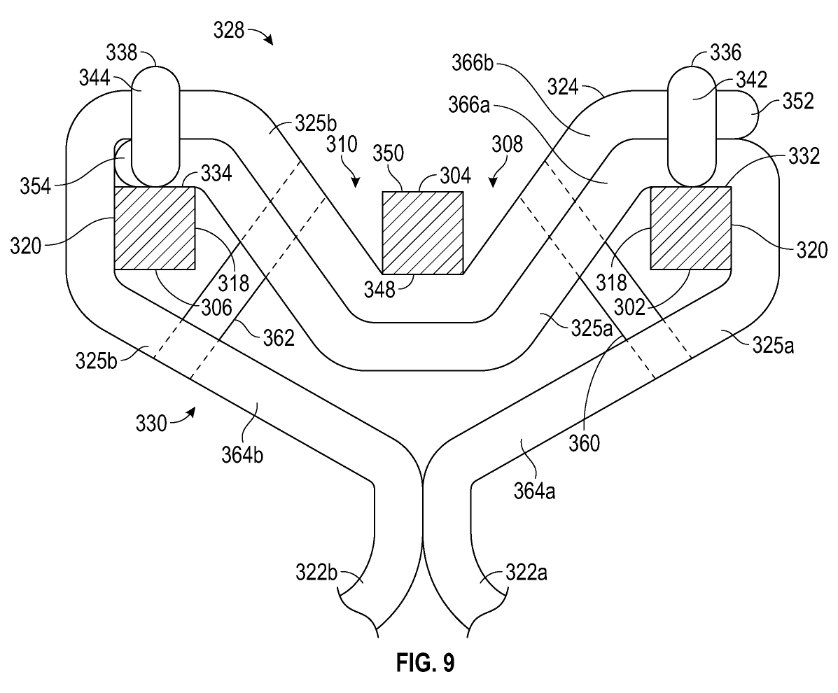

FIG. 9 shows a top plan view of the commissure similar to FIG. 6, but includes different stitching to secure the commissure 324 to the commissure feature 300. The example of FIG. 9 can be identical to the example of FIG. 6, except for the following differences. The example of FIG. 9 includes stitch lines 360, 362 to secure the inner layers of the commissure tabs to adjacent outer layers. In particular, the stitch line 360 can be formed from one or more fasteners (for example, sutures) forming a plurality of in-and-out stitches that extend through an inner layer 364a of the commissure tab 325a, the window 308, an outer layer 366a of the commissure tab 325a, and an outer layer 366b of the commissure tab 325b. The stitch line 360 can comprise a plurality of continuous in-and-out stitches that extend along the height of the commissure window 308. Similarly, the stitch line 362 can be formed from one or more fasteners (for example, sutures) forming a plurality of in-and-out stitches that extend through an inner layer 364b of the commissure tab 325b, the window 310, the outer layer 366a of the commissure tab 325a, and the outer layer 366b of the commissure tab 325b. The stitch line 362 can comprise a plurality of continuous in-and-out stitches that extend along the height of the commissure window 310.

The stitch lines 360, 362 secure the inner layers 364a, 364b in close proximity to the inner surface of the commissure feature 300 and perform a similar function to stitch line 340 (FIG. 6). Therefore, the stitch line 340 need not be included in the example of FIG. 9. In other examples, the commissure 324 can include stitch lines 360, 362, as well as stitch line 340. Although not shown, the example of FIG. 9 can include optional stitch line 370.

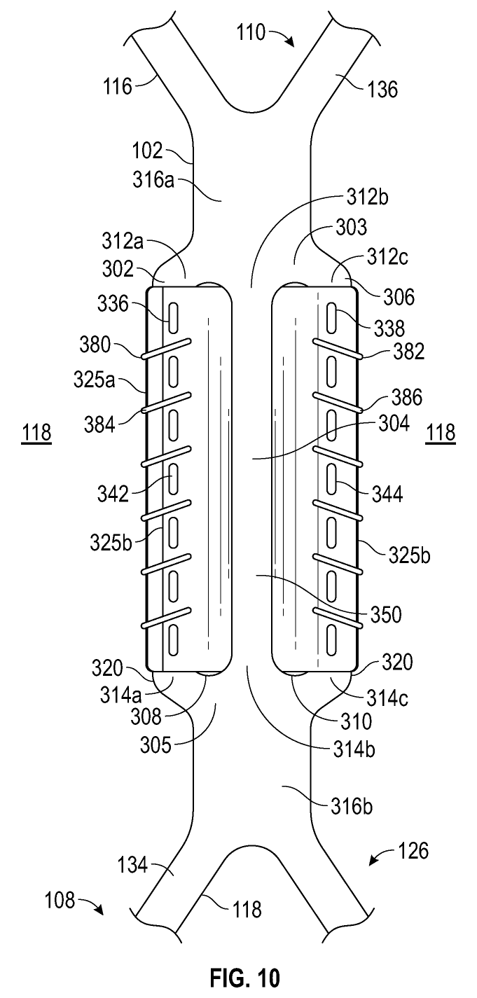

FIG. 10 is a view of the commissure 324 similar to FIG. 7, but includes different stitching to secure the commissure 324 to the commissure feature 300. The example of FIG. 10 can be identical to the example of FIG. 7, except for the following differences. The example of FIG. 10 includes stitch lines 380, 382 to secure the inner layers of the commissure tabs to adjacent outer layers. In particular, the stitch line 380 can be formed from one or more fasteners (for example, sutures) forming a plurality of loop or whip stitches 384 that extend around side strut 302, through an inner layer 364a of the commissure tab 325a, the window 308, an outer layer 366a of the commissure tab 325a, and an outer layer 366b of the commissure tab 325b. The stitch line 380 can comprise a plurality of continuous whip stitches that extend along the height of the strut 302. Similarly, the stitch line 382 can be formed from one or more fasteners (for example, sutures) forming a plurality of loop or whip stitches 384 that extend around side strut 306, through an inner layer 364b of the commissure tab 325b, the window 310, the outer layer 366a of the commissure tab 325a, and the outer layer 366b of the commissure tab 325b. The stitch line 382 can comprise a plurality of continuous whip stitches that extend along the height of the strut 306.

The stitch lines 380, 382 secure the inner layers 364a, 364b in close proximity to the inner surface of the commissure feature 300 and perform a similar function to stitch line 340 (FIG. 6). Therefore, the stitch line 340 need not be included in the example of FIG. 10. In other examples, the commissure 324 can include stitch lines 380, 382, as well as stitch line 340. Although not shown, the example of FIG. 10 can include optional stitch line 370.

Attachment between the commissure 324 and commissure feature 300 as described herein can advantageously secure the commissure 324 and leaflets 322a, 322b to the frame 102 without and/or with limited use of additional sutures or other fasteners extending through and around the struts 302, 304, 306 and/or other portions of the frame 102. This, among other things, allows pairs of adjacent leaflets 322 to be mounted within and properly secured to the frame 102 when constructing the prosthetic heart valve 100 with a relatively small number of stitches conveniently placed on the outside and inside the frame 102. As a result, the act of mounting of the leaflets 322 to the frame 102 can be simplified and less time intensive.

Any of the systems, devices, apparatuses, etc. herein can be sterilized (e.g., with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame comprising a plurality of commissure features, each commissure feature comprising a first sidewall strut, a second sidewall strut, and a central strut situated between the first and second sidewall struts; and a plurality of leaflets arranged within an interior of the frame and secured to one another at adjacent ends to form commissures, each commissure being secured to a respective commissure feature and comprising a first commissure tab of a first leaflet and a second commissure tab of a second leaflet adjacent the first leaflet, wherein the first commissure tab and the second commissure tab each extend inside of the central strut, wrap around a respective sidewall strut, and extend into the interior of the frame.

Example 2. The prosthetic heart valve of any example herein, particularly example 1, wherein the first commissure tab wraps around the first sidewall strut and the second commissure tab wraps around the second sidewall strut.

Example 3. The prosthetic heart valve of any example herein, particularly any one of examples 1-2, wherein the first and second commissure tabs overlap each other outside of the frame.

Example 4. The prosthetic heart valve of any example herein, particularly any one of examples 1-3, wherein the first and second commissure tabs are connected to each other outside of the frame.

Example 5. The prosthetic heart valve of any example herein, particularly any one of examples 1-4, wherein the first and second commissure tabs are connected to each other within the interior of the frame.

Example 6. The prosthetic heart valve of any example herein, particularly any one of examples 1-5, wherein the first and second commissure tabs are sutured to each other.

Example 7. The prosthetic heart valve of any example herein, particularly example 6, wherein the first and second commissure tabs are sutured to one another along first and second stitch lines outside of the frame.

Example 8. The prosthetic heart valve of any example herein, particularly example 7, wherein the first stitch line extends parallel to the first sidewall strut and the second stitch line extends parallel to the second sidewall strut.

Example 9. The prosthetic heart valve of any example herein, particularly any one of examples 7-8, wherein the first and second commissure tabs are sutured to one another along a third stitch line within the interior of the frame.

Example 10. The prosthetic heart valve of any example herein, particularly example 9, wherein the third stich line extends parallel to the central strut.

Example 11. The prosthetic heart valve of any example herein, particularly any one of examples 1-10, wherein the first commissure tab contacts the first sidewall strut and the second sidewall strut.

Example 12. The prosthetic heart valve of any example herein, particularly any one of examples 1-11, wherein the second commissure tab contacts the central strut.

Example 13. The prosthetic heart valve of any example herein, particularly any one of examples 1-12, wherein at least a portion of the first commissure tab is situated between the second commissure tab and the first and second sidewall struts.

Example 14. The prosthetic heart valve of any example herein, particularly any one of examples 1-13, wherein at least a portion of the second commissure tab is situated between the first commissure tab and the central strut.

Example 15. The prosthetic heart valve of any example herein, particularly any one of examples 1-14, wherein an exterior surface of the central strut is exposed.

Example 16. The prosthetic heart valve of any example herein, particularly any one of examples 1-15, wherein the first sidewall strut, the second sidewall strut, and the central strut are circumferentially aligned along a circumference of the frame.

Example 17. The prosthetic heart valve of any example herein, particularly any one of examples 1-16, wherein the first sidewall strut, the second sidewall strut, and the central strut are all equal in length.

Example 18. The prosthetic heart valve of any example herein, particularly any one of examples 1-17, further comprising an outer skirt disposed around an outer surface of the frame.

Example 19. The prosthetic heart valve of any example herein, particularly any one of examples 1-18, wherein the first sidewall strut, the second sidewall strut, and the central strut each comprise a respective upper end, wherein the upper ends of the struts are connected by a first lateral strut.

Example 20. The prosthetic heart valve of any example herein, particularly example 19, wherein the first sidewall strut, the second sidewall strut, and the central strut each comprise a respective lower end, wherein the lower ends of the struts are connected by a second lateral strut.

Example 21. A prosthetic heart valve comprising: an annular frame comprising a plurality of interconnected struts, the plurality of interconnected struts defining a plurality of axially extending commissure features, each commissure feature comprising a first axial strut, a second axial strut, and a central axial strut situated between the first and second axial struts, wherein the first and central axial struts define a first commissure window and the second and central axial struts define a second commissure window; and a plurality of leaflets arranged within an interior of the frame and secured to one another at adjacent ends to form commissures, wherein each commissure is secured to a respective commissure feature by extending laterally across an interior surface of the central axial strut, through the first and second commissure windows, along an exterior surface of the first and second axial struts, and into the interior of the frame.

Example 22. The prosthetic heart valve of any example herein, particularly example 21, wherein at least one pair of adjacent leaflets are coupled to each other on the interior of the frame.

Example 23. The prosthetic heart valve of any example herein, particularly any one of examples 21-22, wherein at least one pair of adjacent leaflets are coupled to each other on the exterior of the frame.

Example 24. The prosthetic heart valve of any example herein, particularly any one of examples 21-23, wherein each commissure comprises a first commissure tab of a first leaflet and a second commissure tab of a second leaflet adjacent the first leaflet, wherein the first and second commissure tabs overlap each other in a radial direction relative to the frame.

Example 25. The prosthetic heart valve of any example herein, particularly example 24, wherein the first commissure tab wraps around the exterior surface of the first axial strut and the second commissure tab wraps around the exterior surface of the second axial strut.

Example 26. The prosthetic heart valve of any example herein, particularly any one of examples 24-25, wherein the first and second commissure tabs are sutured to each other outside of the frame by a first set of stiches and a second set of stiches.

Example 27. The prosthetic heart valve of any example herein, particularly example 26, wherein the first set of stitches is proximate the first axial strut and the second set of stitches is proximate the second axial strut.

Example 28. The prosthetic heart valve of any example herein, particularly example 27, wherein the first set of stitches defines a first stich line parallel to the first axial strut and the second set of stiches defines a second stich line parallel to the second axial strut.

Example 29. The prosthetic heart valve of any example herein, particularly example 28, wherein the first stich line is radially aligned with the first axial strut.

Example 30. The prosthetic heart valve of any example herein, particularly any one of examples 28-29, wherein the second stitch line is radially aligned with the second axial strut.

Example 31. The prosthetic heart valve of any example herein, particularly any one of examples 24-30, wherein the first and second commissure tabs wrap around the exterior of a respective first or second axial strut and extend into the interior of the frame.

Example 32. The prosthetic heart valve of any example herein, particularly example 31, wherein the first and second commissure tabs are sutured to each other within the interior of the frame.

Example 33. The prosthetic heart valve of any example herein, particularly example 32, wherein the first and second commissure tabs are sutured to each other with stitches defining an axially extending stitch line parallel to the central strut.

Example 34. The prosthetic heart valve of any example herein, particularly example 33, wherein the stitch line is radially aligned with the central strut.

Example 35. The prosthetic heart valve of any example herein, particularly example 21-34, wherein the first and second commissure windows are fully enclosed by the commissure tabs.

Example 36. The prosthetic heart valve of any example herein, particularly example 21-35, wherein the first and second commissure windows lie within a circumference of the frame.

Example 37. A prosthetic heart valve comprising: an annular frame comprising a plurality of interconnected struts, the plurality of interconnected struts defining a plurality of adjacent pairs of first and second commissure windows; and a plurality of leaflets arranged within an interior of the frame and secured to one another at adjacent ends to form commissures, each commissure secured to a respective pair of first and second commissure windows by extending outwardly through the first and second commissure windows, around a pair of outer edges of the first and second commissure windows from an exterior of the frame, and inwardly into an interior of the frame from the outer edges of the first and second commissure windows, wherein each commissure comprises a first leaflet tab of one leaflet and a second leaflet tab of an adjacent leaflet secured to one another along first and second stitch lines outside of the frame and a third stitch line inside of the frame.

Example 38. The prosthetic heart valve of any example herein, particularly example 37, wherein each commissure extends circumferentially between the first and second commissure windows inside of the frame.

Example 39. The prosthetic heart valve of any example herein, particularly any one of examples 37-38, wherein the plurality of adjacent pairs of first and second commissure windows are spaced circumferentially from one another around the frame.

Example 40. The prosthetic heart valve of any example herein, particularly any one of examples 37-39, wherein the first and second leaflet tabs overlap in a radial direction relative to the frame along the first and second stitch lines.

Example 41. The prosthetic heart valve of any example herein, particularly any one of examples 37-40, wherein the first and second leaflet tabs overlap in a lateral direction relative to the frame along the third stitch line.

Example 42. The prosthetic heart valve of any example herein, particularly any one of examples 37-41, wherein the first and second leaflet tabs overlap each other between the first and second stitch lines.

Example 43. The prosthetic heart valve of any example herein, particularly any one of examples 37-42, wherein the first and second leaflet tabs only partially overlap between the first stitch line and the third stitch line.

Example 44. The prosthetic heart valve of any example herein, particularly any one of examples 37-43, wherein the first and second leaflet tabs only partially overlap between the second stitch line and the third stitch line.

Example 45. The prosthetic heart valve of any example herein, particularly any one of examples 37-44, wherein the first, second, and third stitch lines are parallel to a longitudinal axis of the frame.

Example 46. The prosthetic heart valve of any example herein, particularly any one of examples 37-45, wherein the first and second stitch lines are radially offset from the third stitch line.

Example 47. The prosthetic heart valve of any example herein, particularly any one of examples 37-46, wherein the first and second stitch lines are circumferentially offset from the third stitch line.

Example 48. The prosthetic heart valve of any example herein, particularly any one of examples 37-47, wherein the first and second commissure windows are defined by a pair of outer struts and an inner strut situated between the outer struts.

Example 49. The prosthetic heart valve of any example herein, particularly example 48, wherein the first leaflet tab only contacts the outer struts of the first and second commissure windows.

Example 50. The prosthetic heart valve of any example herein, particularly any one of examples 48-49, wherein the second leaflet tab only contacts the inner strut and one of the outer struts of the first and second commissure windows.

Example 51. The prosthetic heart valve of any example herein, particularly any one of examples 37-50, wherein for each pair of adjacent first and second commissure windows, the first and second commissure windows are enclosed windows.

Example 52. The prosthetic heart valve of any example herein, particularly any one of examples 37-51, wherein each pair of adjacent first and second commissure windows lies within the circumference of the frame.

Example 53. The prosthetic heart valve of any example herein, particularly any one of examples 1-52, wherein the prosthetic heart valve is sterilized.

Example 54. A method comprising: sterilizing a prosthetic heart valve of any example herein, particularly any one of examples 1-53.

The features described herein with regard to any example can be combined with other features described in any one or more of the other examples, unless otherwise stated. For example, any one or more of the features of one frame can be combined with any one or more features of another frame. As another example, any one or more features of one prosthetic heart valve can be combined with any one or more features of another prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame comprising a plurality of commissure features, each commissure feature comprising a first sidewall strut, a second sidewall strut, and a central strut situated between the first and second sidewall struts; and
a plurality of leaflets arranged within an interior of the frame and secured to one another at adjacent ends to form commissures, each commissure being secured to a respective commissure feature and comprising a first commissure tab of a first leaflet and a second commissure tab of a second leaflet adjacent the first leaflet, wherein the first commissure tab and the second commissure tab each extend inside of the central strut, wrap around a respective sidewall strut, and extend into the interior of the frame.

2. The prosthetic heart valve of claim 1, wherein the first commissure tab wraps around the first sidewall strut, the second commissure tab wraps around the second sidewall strut, and the first and second commissure tabs overlap each other outside of the frame.

3. The prosthetic heart valve of claim 1, wherein the first and second commissure tabs are connected to each other within the interior of the frame.

4. The prosthetic heart valve of claim 1, wherein the first and second commissure tabs are sutured to each other along first and second stitch lines outside of the frame.

5. The prosthetic heart valve of claim 4, wherein the first stitch line extends parallel to the first sidewall strut and the second stitch line extends parallel to the second sidewall strut.

6. The prosthetic heart valve of claim 4, wherein the first and second commissure tabs are sutured to one another along a third stitch line within the interior of the frame.

7. The prosthetic heart valve of claim 1, wherein at least a portion of the first commissure tab is situated between the second commissure tab and the first and second sidewall struts.

8. The prosthetic heart valve of claim 1, wherein at least a portion of the second commissure tab is situated between the first commissure tab and the central strut.

9. The prosthetic heart valve of claim 1, wherein the first sidewall strut, the second sidewall strut, and the central strut each comprise a respective upper end, wherein the upper ends of the struts are connected by a first lateral strut.

10. The prosthetic heart valve of claim 9, wherein the first sidewall strut, the second sidewall strut, and the central strut each comprise a respective lower end, wherein the lower ends of the struts are connected by a second lateral strut.

11. A prosthetic heart valve comprising:
an annular frame comprising a plurality of interconnected struts, the plurality of interconnected struts defining a plurality of axially extending commissure features, each commissure feature comprising a first axial strut, a second axial strut, and a central axial strut situated between the first and second axial struts, wherein the first and central axial struts define a first commissure window and the second and central axial struts define a second commissure window; and
a plurality of leaflets arranged within an interior of the frame and secured to one another at adjacent ends to form commissures, wherein each commissure is secured to a respective commissure feature by extending laterally across an interior surface of the central axial strut, through the first and second commissure windows, along an exterior surface of the first and second axial struts, and into the interior of the frame.

12. The prosthetic heart valve of claim 11, wherein each commissure comprises a first commissure tab of a first leaflet and a second commissure tab of a second leaflet adjacent the first leaflet, wherein the first and second commissure tabs overlap each other in a radial direction relative to the frame.

13. The prosthetic heart valve of claim 12, wherein the first commissure tab wraps around the exterior surface of the first axial strut and the second commissure tab wraps around the exterior surface of the second axial strut.

14. The prosthetic heart valve of claim 12, wherein the first and second commissure tabs are sutured to each other outside of the frame by a first set of stiches and a second set of stiches.

15. A prosthetic heart valve comprising:
an annular frame comprising a plurality of interconnected struts, the plurality of interconnected struts defining a plurality of adjacent pairs of first and second commissure windows; and
a plurality of leaflets arranged within an interior of the frame and secured to one another at adjacent ends to form commissures, each commissure secured to a respective pair of first and second commissure windows by extending outwardly through the first and second commissure windows, around a pair of outer edges of the first and second commissure windows from an exterior of the frame, and inwardly into an interior of the frame from the outer edges of the first and second commissure windows, wherein each commissure comprises a first leaflet tab of one leaflet and a second leaflet tab of an adjacent leaflet secured to one another along first and second stitch lines outside of the frame and a third stitch line inside of the frame.

16. The prosthetic heart valve of claim 15, wherein each commissure extends circumferentially between the first and second commissure windows inside of the frame.

17. The prosthetic heart valve of claim 15, wherein the first and second leaflet tabs overlap in a lateral direction relative to the frame along the third stitch line.

18. The prosthetic heart valve of claim 15, wherein the first and second leaflet tabs overlap each other between the first and second stitch lines.

19. The prosthetic heart valve of claim 15, wherein the first and second leaflet tabs only partially overlap between the first stitch line and the third stitch line.

20. The prosthetic heart valve of claim 15, wherein the first and second leaflet tabs only partially overlap between the second stitch line and the third stitch line.
